(19) United States
(12) Patent Application Publication
(10) Pub. No.: US 2023/0285311 A1
(43) Pub. Date: Sep. 14, 2023

(54) COMPOSITION COMPRISING NANOPARTICLES, METHOD FOR THE PREPARATION OF A COMPOSITION COMPRISING NANOPARTICLES AND USES OF THE COMPOSITION FOR DENTAL TREATMENT

(71) Applicants: Universitat Internacional De Catalunya, Fundacio Privada, Barcelona (ES); Universitat De Barcelona, Barcelona (ES)

(72) Inventors: Fernando Salvador Duran-Sindreu Terol, Barcelona (ES); Jose Antonio Gonzalez Sanchez, Sant Cugat del Valles (ES); Roman Perez Antonanzas, Gava (ES); Firas Elmsmari, Barcelona (ES); Elena Sanchez Lopez, Barcelona (ES); Maria Luisa Garcia Lopez, Barcelona (ES)

(73) Assignees: Universitat Internacional De Catalunya, Fundacio Privada, Barcelona (ES); Universitat De Barcelona, Barcelona (ES)

(21) Appl. No.: 18/001,482

(22) PCT Filed: Jun. 11, 2021
(86) PCT No.: PCT/EP2021/065768

§ 371 Date: Dec. 12, 2022

(30) Foreign Application Priority Data
Jun. 11, 2020 (EP) 20382504.7

(51) Int. Cl. A61K 9/51

(52) U.S. Cl. CPC A61K 9/5153; A61K 9/5115

(57) ABSTRACT

A composition comprising nanoparticles, a method for the preparation of a composition comprising nanoparticles and uses of the composition for dental treatment are provided. Each nanoparticle is made of polymeric material and includes an antimicrobial agent encapsulated therein. The polymeric material can include Poly(lactic-co-glycolic acid) (PLGA), or other polymers such as Polylactic (PLA), Polyglycolic (PGA), or Polycaprolactone (PCL). The active molecule is calcium hydroxide in the range of 0.1 to 5 mg/ml. The composition has a pH in the range of 8-13.

### FIELD OF THE INVENTION

The present invention relates to a composition, in particular for antibacterial dental treatment, comprising polymeric nanoparticles. The invention also relates to a method for the preparation of such composition and uses of the composition for dental treatment, in particular for an endodontic treatment.

### BACKGROUND OF THE INVENTION

The use of nanoparticles is a constantly expanding field and plays a very important role in the medical field. In recent years, nanoparticles are being commonly used in drug administration (drug delivery applications), since these systems allow to direct a wide variety of molecules to different tissues releasing them sustainably over time.

In dental applications, successful prognosis in endodontics is about 85% when a pulp infection exists; therefore, it is necessary to establish mechanisms for increasing the success rate. One of the means to achieve it is by using antibacterial intra-canal therapies, among which is the use of calcium hydroxide. However, calcium hydroxide products used up to now present limitations such as a buffer effect or a non-controlled release, quite possibly due to the structural composition thereof.

Some patents and/or patent applications are known which use nanoparticles for dental treatment. For example, from U.S. Pat. No. 8,105,086 a biomaterial for a dental and medical treatment and its use for sealing and/or filling the tooth and bone are known. The calcium salt, calcium oxide, calcium silicate and calcium phosphate compounds are mixed with a water-based solution, and a mixture enriched with bioactive phosphate and calcium is prepared. The mixture, in the form of nanoparticles, comprises a high concentration of water-soluble calcium and phosphate and, accordingly, forms hydroxyapatite during and after the setting. The biomaterial is biocompatible, antibacterial and is capable of forming an effective seal against the re-entry of microorganisms in the cavity.

Likewise, microparticles for a medical treatment are also known in the state of the art. For example, in patent application US 2017135960, a solid microparticle (for example, non-porous) is disclosed, for ocular therapy in particular, loaded with a drug. Processes are also described for producing the superficially-treated microparticle and injectable formulations which include the superficially-treated microparticle.

Likewise, patent KR 101879399 refers to a method for producing polymeric microparticles using dialkyl carbonate (a non-halogenated organic solvent for solubilizing a polymeric compound). The microparticles can efficiently enclose drugs at various concentrations.

Moreover, in the document “PLGA-based nanoparticles for sustained release of Ca++ for apexification”, B. I. Cerda-Cristerna et al. Abstracts of the Academy of Dental Materials Annual Meeting, 12-15 Oct. 2016—Chicago, USA, nanoparticles of Poly(L(+)-lactic acid-co-glycolic) or PLGA for the sustained release of Ca++ for apexification are presented. These PLGA nanoparticles, unlike the present invention, do not present a percentage of efficiency of association of the active compound to the nanoparticles; therefore the authors do not present any confirmation that the nanoparticles include the active principle (CaOH) encapsulated. Furthermore, as indicated in the study, the nanoparticles used have an initial neutral pH of 7.51 and an acid pH at the end of the experiment of 2.44. Therefore, due to this acidification of the nanoparticles over time, they cannot be used in dental disinfection treatments.

Document Cerda-Cristerna et al. “Sustained release of calcium hydroxide from poly(DL-lactide-co-glycolide) acid microspheres for apexification”, Odontology 2016, Sep. 104(3):318-23 discloses a study formulating and characterizing calcium hydroxide-PLGA microspheres. Said document describes a method form making PLGA particles encapsulating Ca(OH)₂ using an O/W technique and emulsion solvent evaporation technique. Document Xiaoman et al. “Chitosan-decorated calcium hydroxide microcapsules with pH-triggered release for endodontic applications”, J Mater Chem B. 2015 Dec. 7; 3(45):8884-8891 discloses calcium hydroxide microcapsules coated with chitosan and ethylcellulose. Drug release from the microcapsules is pH-triggered.

Therefore, there are no known nanoparticles of polymeric material, particularly Poly(L(+)lactic acid) or PLGA, which include encapsulated calcium hydroxide as an antimicrobial agent and that maintain a high pH over time.

Hence, new compositions are required for dental treatment that includes such nanoparticles and new methods for preparing the nanoparticles.

### DISCLOSURE OF THE INVENTION

To that end, exemplary embodiments of the present invention provide, according to a first aspect, a composition which comprises nanoparticles. The nanoparticles are formed by a polymeric material and include an antimicrobial agent, particularly calcium hydroxide. According to the present invention, the polymeric material is selected from the group comprising PLGA, polylactic acid (PLA), polyglycolic acid (PGA), polycaprolactone (PCL) and/or combinations thereof. The calcium hydroxide is encapsulated in the nanoparticles and is in the range from 0.1 to 5 mg/ml. Furthermore, the composition has a pH in the range of 8-13. This pH is beneficial because calcium hydroxide has antimicrobial activity due to its basic pH.

By using the referred nanoparticles, the present invention allows to control the dosage, the release and the target penetration of the calcium hydroxide.

In some embodiments, the nanoparticles can include calcium hydroxide on its surface.

In an embodiment, the polymeric material is PLGA. The PLGA can be comprised in a range from 5 to 20 mg/ml in said composition. Preferably, the PLGA will be comprised between 10 and 12 mg/ml.

In particular, the composition/formulation is in an aqueous solution. However, in an embodiment, it can be lyophilized, so that it may be resuspended at the time of use, thereby increasing its stability.

The nanoparticles can have a size between 150 and 300 nanometers. In particular, the size of the nanoparticle is about 150 nanometers. This way, irritation phenomena, collapse of filters or other materials are avoided and the correct encapsulation and the sustained release of the calcium hydroxide are guaranteed.

In an embodiment, the nanoparticles have a polydispersity index lower than 0.2 and a negative surface charge.

According to the present invention, the polymeric material can be biodegradable. Likewise, the composition can be in the form of a gel, an aqueous solution or it can be in the form of dispersed particles.

Embodiments of the present invention also provide, according to a second aspect, a method for preparing a composition comprising nanoparticles. The method comprises:
preparing an organic phase comprising adding a first mixture, including calcium hydroxide and a first solvent, to a second mixture, including a polymeric material and a second solvent material, wherein the polymeric material can include PLGA, PLA, PGA, PCL, or even combinations thereof;
preparing an aqueous phase comprising a surfactant product and leaving the aqueous phase under magnetic stirring at a certain stirring speed;
adjusting a pH of the aqueous phase to a value above 10;
adding the organic phase into the aqueous phase, drop by drop at a constant rate during a first predetermined time, wherein the mixture is left under magnetic stirring during a second predetermined time;
carrying out a low-pressure evaporation process for removing said first and second solvent materials; and
obtaining the nanoparticles.

In an embodiment, the method also comprises lyophilizing the obtained nanoparticles. That is, extracting the water from the nanoparticles by a sublimation process, thus increasing the stability of the nanoparticles.

In an embodiment, the first solvent comprises dimethyl sulfoxide (DMSO) and the second solvent comprises acetone. In an exemplary embodiment, the method comprises using 1 ml of DMSO and 4 ml of acetone.

In an embodiment, the calcium hydroxide is comprised in a range from 0.1 to 5 mg/ml in the first mixture and the polymeric material, PLGA in particular, is comprised in a range from 5 to 20 mg/ml in the second mixture. Likewise, the surfactant is comprised in a range from 5 to 15 mg/ml.

The constant drop rate of the organic phase into the aqueous phase can vary between 2 and 5 seconds, preferably 3 seconds. Likewise, each drop has a volume of 50 microliters.

In an embodiment, the pH value of the aqueous phase is adjusted to a value about 11-12.

In a particular exemplary embodiment, the calcium hydroxide is present in a concentration of 1.7 mg/ml, the polymeric material (PLGA) in 11.5 mg/ml and the surfactant (Poloxamer 188) in 11 mg/ml dissolved in a aqueous buffer prepared at pH 12. By means of this process, a nanoparticle average size of 199.9±1.35 nm is obtained. Likewise, the nanoparticles, in this particular exemplary embodiment, have a polydispersity index of 0.174±0.021; a zeta potential of −6.41±0.10 mV and an association efficiency of 24.98%.

In a third aspect of the invention, there is provided a use of the composition according to the invention for the following dental treatments: as an antibacterial compound for its placement inside the root canal, as a sealant of the root canal, for pulp capping, for repairing the dentin or dental-periodontal communication, as a material for the retrograde obturation in an apical surgery, as indirect pulp capping and as cavitary base.

### BRIEF DESCRIPTION OF THE DRAWINGS

The foregoing and other characteristics and advantages will be more fully understood from the following detailed description of some exemplary embodiments, merely illustrative and non-limiting, with reference to the attached drawings, wherein:

FIG. 1 schematically depicts a method for preparing the nanoparticles, PLGA in this case, encapsulating calcium hydroxide as an active molecule or antimicrobial agent, according to an embodiment of the present invention.

FIG. 2 schematically depicts the process of filtration-centrifugation for quantifying non-encapsulated calcium hydroxide.

### DETAILED DESCRIPTION OF THE INVENTION AND OF EXEMPLARY EMBODIMENTS

The present invention provides a composition, particularly for antibacterial dental treatment, comprising nanoparticles of biodegradable polymeric material, for example PLGA, PLA, PGA, PCL, or combinations thereof. In particular, the nanoparticles are of PLGA.

The nanoparticles are of nanospherical type with encapsulated calcium hydroxide. The composition comprising the nanoparticles has a pH value in the range of 8-13, preferably 9-13, more preferably 10-13.

Particularly, the nanoparticles are also enclosed by a surfactant material and comprise a certain quantity of non-encapsulated calcium hydroxide which is located around the surface of each nanoparticle. Subsequent to the method for preparing the nanoparticles, an additional quantity of Ca(OH)₂ is added as well as 5% of hydroxpropyl-β-cyclodextrin. Optionally, 15% of mannitol may also be added.

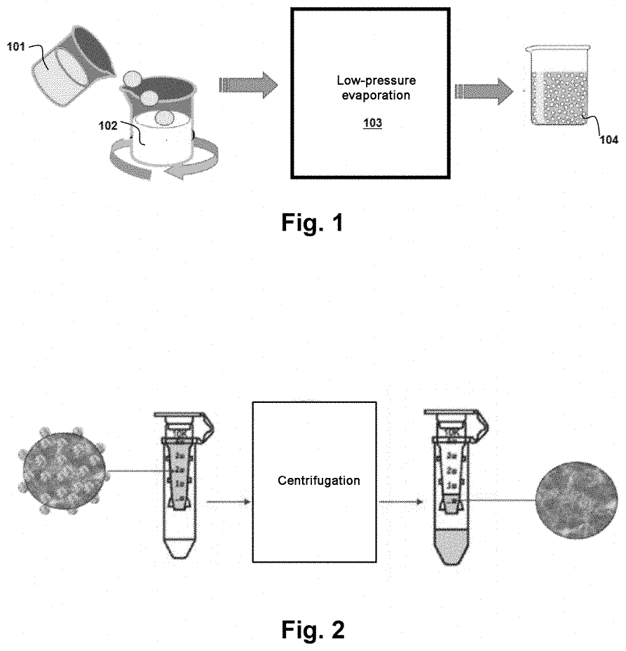

With reference to FIG. 1, therein an embodiment of the displacement method of the solvent used for elaborating the nanoparticles, PLGA in this case, is illustrated. To that end, in a first phase or organic phase 101, the method comprises separately preparing:
calcium hydroxide in 1 ml of anhydrous DMSO; and
poly(lactic-co-glycolic acid) or PLGA in 4 ml of acetone.

Subsequently, DMSO containing the calcium hydroxide is added to the 4 ml of acetone.

Thereafter, the second phase or aqueous phase 102 is prepared. According to this embodiment, this phase contains Poloxamer 188 (BASF Chemicals) as surfactant in a concentration of about 11 mg/ml. This aqueous phase is made of a buffer solution adjusted to pH 12. The aqueous phase is left under magnetic stirring adding a magnetic core at 400 r.p.m. per minute.

Next, the organic phase 101 is added to the aqueous phase 102, which is under magnetic stirring. The addition is carried out dropwise at a constant rate, adding, for example, a drop in the center of the stirring cone every 3 seconds. The total time this process takes is approximately 5 minutes.

Then, the mixture is left under magnetic stirring for another 10 minutes. Afterwards, it is evaporated at a reduced pressure 103 (Buchi rotavapor), 9 kPa, for 20 minutes, approximately, to remove the organic solvents used. 10 ml of the formulation of dispersed nanoparticles in water 104 are obtained.

In an embodiment, to achieve greater stability, the obtained nanoparticles are lyophilized. Lyophilizing consists in the extraction of water from the sample by means of a sublimation process. To that end, substances (cryoprotectants) which protect the nanoparticles against the aggression of the process are added and it consists of three steps: freezing, primary drying (here, the water of the nanoparticle is sublimated by using the low pressures), secondary drying (temperature is increased up to 20 degrees Celsius, leaving the pressure of the previous step constant, thereby also evaporating the structural water of the nanoparticles). Advantageously in the present invention it has been proved that after the lyophilization the nanoparticles maintain their initial physical-chemical characteristics. It has been found that by re-suspending the nanoparticles in a liquid medium after lyophilization, the original pH value is obtained, i.e. between 8 and 13.

The key parameters in the composition/formulation have been studied by creating a design space. To that end, the optimization has been carried out by means of a central factorial design created by the Statgraphics Centurion programme. Therein, the concentration parameters of the indicated compounds are optimized, as well as the pH of the aqueous phase 102. The influence in the average size, the polydispersity index, the Zeta potential (carried out by means of laser-Doppler electrophoresis with the M3 PALS system) and the calcium hydroxide association efficiency to the nanoparticles are studied. Thus, the tendencies the nanoparticles follow, as well as the corresponding response superficies, are obtained, which is necessary for achieving a definitive formulation guaranteeing that it is the best formulation that can be obtained.

The optimized nanoparticles present an average size lower than 300 nm, a polydispersity index lower than 0.2, characteristic of the unimodal systems, and a negative surface charge (approximately of −20.0 mV). The polydispersity index is analyzed with a zetasizer model Zetasizer Nano ZS by means of the dynamic light scattering technique. Additionally, the purpose is to encapsulate the maximum quantity possible of calcium hydroxide within the nanoparticles. The encapsulation efficiency (EE) of the nanoparticles is measured indirectly by means of the separation of the encapsulation-free drug by means of filtration-centrifugation (FIG. 2). The fraction of free calcium hydroxide is quantified and, using the following equation, the encapsulated quantity is derived:

$$EE ( \% ) = \frac{\begin{matrix}Initial calcium hydroxide concentration - \\ F \overline{ree calcium hydrox} ide concentration\end{matrix}}{Initial calcium hydroxide concentration} \cdot 100$$

For the characterization of the nanoparticles, the drug-polymer interactions have been studied using various analysis techniques such as infrared spectroscopy (FTIR), differential scanning calorimetry (DSC) and X-ray diffraction of the freeze-dried samples. Additionally, the liquid samples have been visualized by means of transmission electronic microscopy (TEM) after a negative staining.

Likewise, in some embodiments, it has been proven by confocal microscopy that the nanoparticles penetrate more than the free drug.

### Example: pH Stability

To test pH stability of the composition according to the present invention, nanoparticles comprising calcium hydroxide and PLGA were studied. An aqueous buffer at pH 12 was prepared, by preparing 100 mL of 0.05 M Na₂HPO₄, 53.8 mL of 0.1 M NaOH and adding water up to 200 mL. Poloxamer 188 was solved in 10 mL of the previously mentioned buffer obtaining a concentration of 11 mg/mL. The organic phase was prepared by mixing Calcium hydroxide with 1 mL of DMSO and PLGA with 4 ml of acetone. Afterwards, both were mixed obtaining an organic phase. This organic phase was added dropwise on the water phase under magnetic stirring. Afterwards, the organic solvents were evaporated under reduced pressure. The final concentration of calcium hydroxide was 1.7 mg/mL and 11.5 mg/mL of PLGA.

The aqueous buffer had an initial pH of 12, and pH of the composition according to the invention was measured using a pH meter for a total of 18 days, yielding values over 7.5 during the entire measurement scope. The following table shows the values of the composition from 0 to 432 hours.

| Time (hours) | pH |
|---|---|
| 0 | 9.65 |
| 0.5 | 9.49 |
| 1 | 9.45 |
| 2 | 9.38 |
| 3 | 9.38 |
| 5 | 9.20 |
| 24 | 9.12 |
| 27 | 9.05 |
| 48 | 9.72 |
| 72 | 9.80 |
| 168 | 8.72 |
| 192 | 8.38 |
| 216 | 8.25 |
| 240 | 8.17 |
| 288 (12 days) | 8.05 |
| 336 (14 days) | 7.92 |
| 384 (16 days) | 7.78 |
| 432 (18 days) | 7.77 |

Such as they are used in this document, the terms “about” and/or “approximately”, when they refer to a value or characteristic, are meant to cover variations of ±10% of some embodiment, ±5% of some embodiment, relative to the specified value or characteristic, as said variations are appropriate to carry out the described composition and method/uses.

Although in this document the embodiments of the present invention have been described with reference to various specific features, it will be obvious for a skilled person to carry out the invention with modifications. All of these modifications are considered to be within the scope of the claims. Likewise, the claims are intended to cover all the generic and specific characteristics of the described exemplary embodiments and all the statements of the scope of protection which, by language reasons, may be said to be therewithin.

The scope of the present invention is set forth in the attached claims.

What is claimed is:

1. A composition comprising nanoparticles, the nanoparticles comprising a polymeric material and an antimicrobial agent comprising calcium hydroxide, wherein:
the polymeric material is selected from the group consisting of: poly(L(+)-lactic-co-glycolic acid), PLGA; polylactic acid, PLA; polyglycolic acid, PGA; polycaprolactone, PCL; and/or combinations thereof;
the antimicrobial agent being encapsulated in the nanoparticles, the antimicrobial agent comprising a concentration between 0.1 mg/ml to 5 mg/ml; and
the composition comprising a pH between 8 and 13.

2. The composition according to claim 1, wherein the nanoparticles comprise a size lower than 300 nm.

3. The composition according to claim 1, wherein the polymeric material is PLGA comprising a concentration between 5 mg/ml to 20 mg/ml.

4. The composition according to claim 1, wherein the nanoparticles have a polydispersion index lower than 0.2 and a negative surface charge.

5. The composition according to claim 1, wherein the polymeric material is biodegradable.

6. The composition according to claim 1, wherein the composition is a gel, an aqueous solution or is in the form of dispersed particles.

7. A method for the preparation of a composition comprising nanoparticles, the method comprising the following steps:
preparing an organic phase that comprises adding a first mixture to a second mixture, wherein the first mixture comprises calcium hydroxide and a first solvent and the second mixture comprises a polymeric material and a second solvent, the polymeric material is selected from the group consisting of: poly(L(+)-lactic-co-glycolic acid), PLGA; polylactic acid, PLA; polyglycolic acid, PGA; polycaprolactone, PCL; and/or combinations thereof;
preparing an aqueous phase comprising a surfactant product and placing the aqueous phase under a magnetic stirring;
adjusting a pH of the aqueous phase to a value above 10;
adding the organic phase into the aqueous phase, drop by drop at a constant rate, and leaving a resulting mixture under the magnetic stirring;
conducting an evaporation process at low pressure to remove the first solvent and second solvent; and
obtaining the nanoparticles.

8. The method according to claim 7, further comprising lyophilizing the obtained nanoparticles.

9. The method according to claim 7, wherein the first solvent comprises dimethyl sulfoxide, DMSO, and the second solvent comprises acetone.

10. The method according to claim 9, wherein the second solvent comprises a volume that is 4 times the volume of the first solvent.

11. The method according to claim 7, wherein the polymeric material comprises PLGA, and wherein a quantity of the calcium hydroxide is double than the quantity of the organic phase relative to a concentration of the nanoparticles and a quantity of PLGA is double relative to the concentration of the nanoparticles.

12. The method according to claim 7, wherein the pH value is adjusted to a value that is between 11 and 12.

13. The method according to claim 7, wherein the drop rate of the organic phase into the aqueous phase is between 2 and 5 seconds.

14. The method according to claim 7, wherein each drop has a volume of 50 microliters.

15. The composition according to claim 1 for use in dental treatment as an antibacterial compound for its placement inside of a root canal, as a sealant of the root canal, for pulp capping, for repairing a dentin or dental-periodontal communication, as material for retrograding obturation in apical surgery and/or as indirect pulp capping and as cavitary base.

16. The composition according to claim 1, wherein the nanoparticles comprise a size that is lower than 150 nm.

17. The composition according to claim 1, wherein the polymeric material is PLGA comprising a concentration between 10 mg/ml to 12 mg/ml.

18. The method according to claim 7, wherein the drop rate of the organic phase into the aqueous phase is 3 seconds.
